(19) United States
(12) Patent Application Publication
(10) Pub. No.: US 2017/0367829 A1
(43) Pub. Date: Dec. 28, 2017

(54) METHOD AND APPARATUS FOR MAKING JOINT PROSTHESIS

(71) Applicant: Concepto LLC, Cincinnati, OH (US)

(72) Inventors: Sambhu N. Choudhury, Cincinnati, OH (US); Suresh Nayak, Cincinnati, OH (US)

(21) Appl. No.: 15/617,073

(22) Filed: Jun. 8, 2017

Related U.S. Application Data
(60) Provisional application No. 62/347,360, filed on Jun. 8, 2016.

(51) Int. Cl. A61F 2/30; A61F 2/36; A61L 27/54; A61L 24/00

(52) U.S. Cl. CPC A61F 2/30965; A61L 24/0015; A61F 2/3607; A61L 27/54; A61F 2002/30975; A61F 2310/00353; A61L 2300/406; A61L 2430/24

(57) ABSTRACT

A method and apparatus are provided for making a temporary hip joint prosthesis. The method includes horizontally positioning each half of a two-part mold having a respective impression of a front side and a backside of a hip joint prosthesis. The method includes level filling the selected impressions with a bone cement mixture. The method includes rotating one half of the two-part mold to align the front side and back side of the hip joint prosthesis. The method includes maintaining the two halves of the two-part mold in contact as portions of the bone cement mixture in each half adhere together and cures. The method includes removing a hip joint prosthesis from the two-part mold.

### PRIORITY AND CROSS-REFERENCE TO RELATED APPLICATIONS

This application claims the benefit of U.S. Provisional Application No. 62/347,360, entitled “METHOD AND APPARATUS FOR MAKING JOINT PROSTHESIS” and filed 8 Jun. 2016, the contents of which is hereby incorporated by reference in its entirety.

### FIELD OF THE INVENTION

The field of art disclosed herein pertains to forming prosthetic hip implant devices, and more particularly to making molded hip implant devices.

### BACKGROUND OF THE INVENTION

Total hip replacement surgery is commonly performed to alleviate pain and loss of function in injured and diseased hip joints. During this surgery, the articulating surfaces of the hip joint are replaced with prosthetic bearing components. The replacement components generally include a femoral component having a convex bearing surface.

In some instances, an infection occurs in the tissue and bone surrounding the prosthesis. In order to treat the infection without the benefit of use of the affected limb, a temporary prosthesis with an incorporated antibiotic compound can be implanted. Due to the variability in the types of infections and shelf life of antibiotics, a need exits to create these temporary prosthesis devices close to the time of implantation.

### SUMMARY OF THE INVENTION

In one aspect, the present disclosure provides a method for making a temporary hip joint prosthesis. The method includes horizontally positioning each half of a two-part mold having a respective impression of a front side and a backside of a hip joint prosthesis. The method includes level filling the selected impressions with a bone cement mixture. The method includes rotating one half of the two-part mold to align the front side and back side of the hip joint prosthesis. The method includes maintaining the two halves of the two-part mold in contact as portions of the bone cement mixture in each half adhere together and cures. The method includes removing a hip joint prosthesis from the two-part mold.

In one or more embodiments, a coupling mechanism joins the first mold to the second mold such that the cement mold is substantially sealed to define the implant.

In one or more embodiments, an input port is defined by the cement mold and is operable to receive a delivery nozzle to supply antibiotic loaded bone cement within the inner sidewall.

In one or more embodiments, at least one ventilation port is defined by the cement mold and is operable to vent trapped air upon filling the cement mold with the antibiotic loaded bone cement through the input port.

In one or more embodiments, a removal mechanism forming a portion of the cement mold is operable to assist in separating the cement mold from the implant.

In one or more embodiments, a method for forming a implant includes mixing bone cement during a surgical procedure, selecting an appropriately sized cement mold that is translucent, filling the cement mold with the bone cement to form the implant, determining that the cement mold is filled by viewing through the translucent cement mold, and implanting the implant into a patient.

In one or more embodiments, a method of forming a implant includes mixing bone cement during a surgical procedure, selecting an appropriately sized cement mold having an input port, filling a cement gun having a nozzle with the bone cement, inserting the nozzle of the cement gun into the input port of the cement mold, filling the cement mold with bone cement by way of directing the nozzle within the cement mold to substantially fill the cement mold, and implanting the implant into a patient.

In one or more embodiments, a method of forming a implant includes mixing bone cement during a surgical procedure, selecting an appropriately sized cement mold that is pierceable, filling the cement mold with the bone cement to form the implant, piercing the cement mold to relieve an air pocket formed within the cement mold, and implanting the implant into a patient.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 illustrates a perspective two of a two-part mold having two sizes of hip implant impressions being filled with reinforcement components and a bone cement mixture, according to one or more embodiments;

FIG. 2 illustrates a top view of the filled two-part mold of FIG. 1 being assembled to cure the two sizes of hip joint prosthesis, according to one or more embodiments;

FIG. 3 illustrates a side view of a smaller hip prosthesis that has been removed from the two-part mold of FIG. 1, according to one or more embodiments;

FIG. 4 illustrates a side view of a larger hip prosthesis that has been removed from the two-part mold of FIG. 1, according to one or more embodiment;

FIG. 5 illustrates a top view of a spacer inserted into the two-part mold of FIG. 1 to provide a selected length of prosthesis;

FIG. 6 illustrates a diagram of a datastructure containing two-part mold impression sizes and spacer lengths and

FIG. 7 illustrates a method of making a temporary hip joint prosthesis, according to one or more embodiments.

### DETAILED DESCRIPTION

The following description is merely exemplary in nature and is not intended to limit the present disclosure, application, or uses. Although certain examples and surgical methods disclosed herein are in conjunction with a temporary hip implant, it is understood that the molds and surgical methods disclosed herein can be used in any orthopedic revision surgery for any area in the patient.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

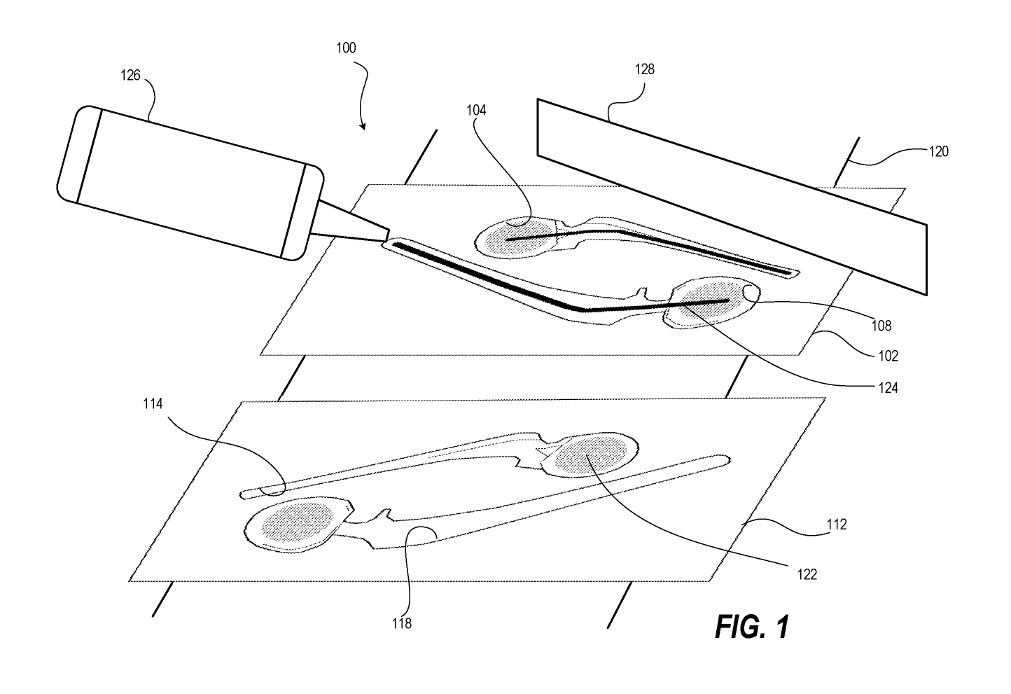

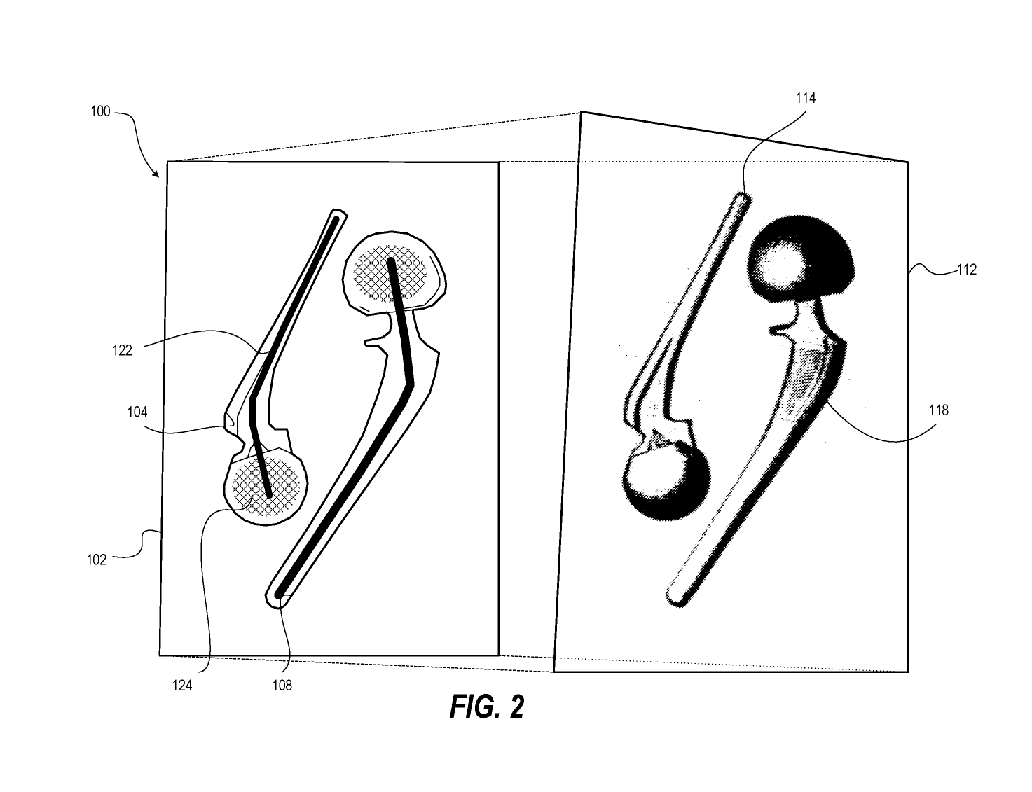

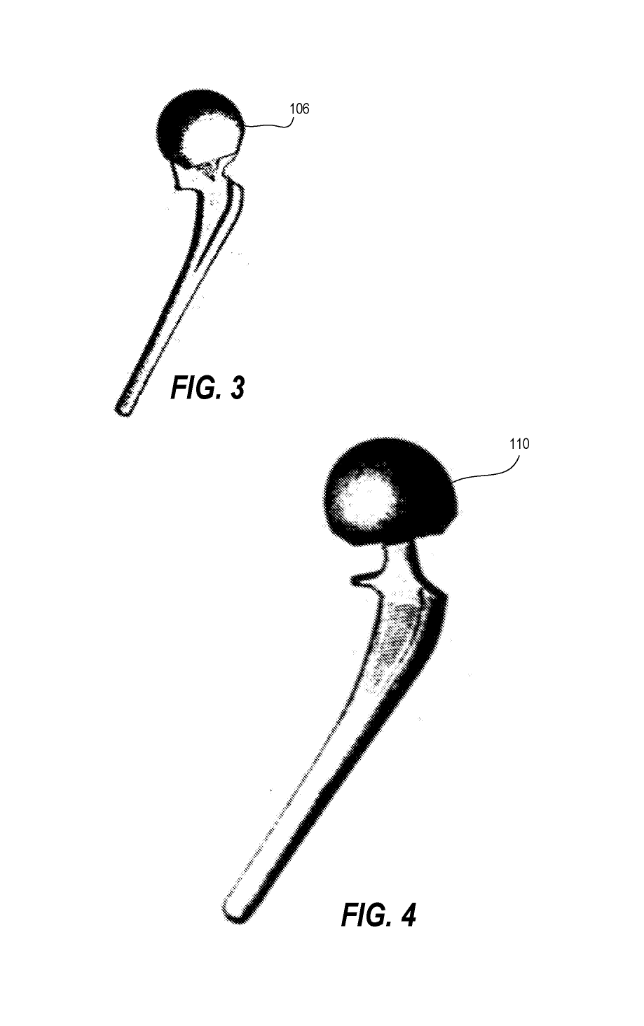

FIGS. 1-2 illustrate a two-part mold 100 having a first half mold 102 with a first impression 104 of a back side of a smaller right hip prosthesis 106 (FIG. 3) formed by using the two-part mold 100. The first half mold 102 also includes a second impression 108 of a backside of a larger right hip prosthesis 110 (FIG. 4) formed by using the two-part mold 100. The two-part mold 100 includes a second half mold 112 with a first impression 114 of a front side of the smaller right hip prosthesis 106 (FIG. 3). The second half mold 112 also includes a second impression 108 of a front side of the larger right hip prosthesis 110 (FIG. 4). The first and second half molds 102, 112 are supported on support tray/s 120 that keep the impressions level and without distortion from the weight of mold contents.

With particular reference to FIG. 1, reinforcing materials such as metal reinforcement rods 122 and metal or synthetic mesh 124 can be placed in the impressions 104, 108, 114, 118 for additional structural strength. A bone cement mixture 126 that includes antibiotic materials and medicines is dispensed into the impressions 104, 108, 114, 118 with any excess removed by a straight edge scraper 128.

In one or more embodiments, with particular reference to FIG. 1, the first impressions 104, 114 and the second impressions 108, 118 respectively of the first and second half molds 102, 112, which are aligned and brought together to form respectively the smaller right hip prosthesis 106 (FIG. 3) and the larger right hip prosthesis 110 (FIG. 4), the inner sidewall which faces the prosthesis is also formed with a plurality of ridges, not shown, to provide additional surface area for leaching of the antibiotic, as well as a textured surface to engage the bone. It should further be noted that ridges or any other texture such as protrusions, dimples or other features creating a textured surface on the spacer may also be applied to any of the appropriate inside surfaces of any of the cement molds discussed herein. Any combination of features and parts as detailed herein can be included in the the first and second half molds 102, 112, such as, for example, an inclusion of a variety of differing surface area increasing features being included on the different components to allow for increased drug elution from the prosthesis 106. In one or more embodiments, one or more drug deliver device(s) may be incorporated into the prosthesis 106.

With particular reference to FIG. 2, the first impressions 104, 114 and the second impressions 108, 118 respectively of the first and second half molds 102, 112 are aligned and brought together to form respectively the smaller right hip prosthesis 106 (FIG. 3) and the larger right hip prosthesis 110 (FIG. 4). Embodiments consistent with aspects of the present innovation can include only one impression in each half of the two-part mold. Alternatively, embodiments consistent with aspects of the present innovation can have more than two impressions. In addition, only selected impressions can be filled. Right side hip prostheses are depicted for clarity.

Referring to FIGS. 1-4, the present teachings provide a modular cement spacer mold for forming a temporary implant. The system includes a head component and a stem component mold, as shown in FIGS. 1-2. It is understood that various features can be interchanged within the scope of the present teachings. The present temporary implants are optimized for strength and reinforce the high stress areas along the neck of the implant. It is understood that the present teachings, while illustrative of a hip implant, can also be used for other orthopedic uses, including, for example, a shoulder or a knee.

The present teachings further provide methods of providing and using the modular cement mold. Although the methods are disclosed as used with certain embodiments of the present teachings, it is understood that the methods disclosed can be used with any of the mold embodiments detailed above herein.

In one or more embodiments, the mold 100 may also contain optional vents in one or all of the mold members. The vents can allow air to escape during the injection of the curable material and can further provide a visual indicator that the mold is full, such as when the curable material begins to extrude out of the vents. The curable material that is used for the curing and forming of the temporary prosthesis can comprise a bone cement material that is typically known in the art, such as a material made out of polymethyl methacrylate (PMMA), or other similar materials. Optionally, an antimicrobial component can be added to the mixture of the curable material to provide a temporary prosthesis that has antimicrobial properties therein. Any known antimicrobial component may be utilized, and in particular, antibiotics such as gentamycin or clindamycin can be used.

As used herein, the term “cement” generally refers to any curing and hardening material suitable for implanted spacers. The cement may be loaded with an antibiotic such as Gentamicin, Vancomycin, Tobramycin and/or Clindamycin in order to clear infection from tissue surrounding an implanted spacer formed by a spacer mold in accordance with the present disclosure. Exemplary cement is described in the “Zimmer® Bone Cement and Accessories” brochure, copyright 2006, published by Zimmer, Inc., the entire disclosure of which is hereby expressly incorporated herein by reference. This includes materials such as Palacos® R+G High Viscosity Bone Cement and any other similar material.

As the uncured bone cement hardens (cures or sets), mold 100 forms a final shape based on various factors including, but not limited to, volume of cement composition added, mold shape, surrounding bone shape, and surgeon contouring. The bone cement can include Simplex™ P brand bone cement with Tobramycin manufactured by Stryker Corp. The Tobramycin reduces infection by killing the bacteria causing the infection. The bone cement composition can be introduced into the interior space through an opening located at an end portion of mold 100.

The bone cement composition can comprise freshly mixed uncured bone cement and an antibiotic. The bone cement can be any bone cement known in the art and can be selected based on the requirements of the particular situation. The antibiotic can be selected based on the particular infection that is present in the femoral cavity. The bone cement composition, usually comprises a liquid monomer and a powdered polymeric component, can be prepared using well known techniques in the art such as a mixer device. Once the cement composition is mixed, it is inserted into a chamber of a dispenser tool such as a syringe or cement gun.

In one or more embodiments, the two-part mold 100 may be made of any suitable mold-making materials that are compatible with the bone cement compositions of the present invention.

In one or more embodiments, the two-part mold 100 comprises at least one first composition and at least one second composition that when mixed, react to form a third composition in the form. The two-part mold 100 according to the present disclosure may directly adhere to an exterior shell or they may be self-contained without the need for an exterior shell. The two-part mold 100 may further comprise a flexible membrane encompassing the at least one first composition and the at least one second composition. The flexible membrane comprises, for example, but is not limited to silicone rubbers such as DUROPRENE, or the silicone rubbers sold under the trade name DRAGON SKIN, such as DRAGON SKIN 10, by Smooth-On, or the resin material sold under the trade name BLUESIL, such as BLUESIL V-340+FC Catalyst, by Bluestar Silicones and distributed by Freeman Manufacturing Y Supply Co. (www.freemansupply.com); polyurethanes; polycaprolactones, synthetic rubbers; and nylon.

The modular cement mold can be formed from any biocompatible material including various polymers. In various embodiments, the polymeric material can be readily tearable and/or translucent, such as a thermoplastic elastomer. In various embodiments, the thermoplastic elastomer is silicone. In such embodiments, the silicone selected can have a sufficiently high stiffness such that the modular cement mold will not sag or be deformed upon handling. Moreover, it should be noted that the material selected should generally not adversely react with the bone cement and antibiotic selected.

Regardless of the material chosen, the flexible membrane is either rigid or pliant but must be sufficiently flexible with a proper tensile strength that it will not rupture under the pressure of the prosthesis, but will flex and allow the encased compositions to harden and to allow removal of the prosthesis.

In accordance with the present invention, the two-part mold 100 for the preparation of a hip prosthesis 106 is made from silicone.

Generally, silicones are inorganic-organic polymers consisting of an inorganic silicon-oxygen backbone (—Si—O—Si—) with organic side groups attached to the silicon atoms. Silicones have many desirable properties, including non-toxicity, thermal stability, freezing resistance, weather resistance, chemical resistance, hot-water resistance, electrical insulation, radiation resistance, oil resistance, pigmentation, transparency, elasticity, and recovery power. The mold for the preparation of recipient blocks in accordance with the present invention retains the above properties of silicones.

In a preferable embodiment, the two-part mold 100 for the preparation of a hip prosthesis 106 in accordance with the present invention is made from silicone rubber. Depending on the degree of polymerization of the material polymer, silicon rubber is classified into millable rubber and liquid silicone rubber. Both of them are useful in the preparation of the mold according to the present invention.

In the case of using millable rubber, silicone polymer (raw rubber) is compounded with silica-based reinforcing fillers and various additives to afford a base compound, which is then heat cured in the presence of a curing agent to form a two-part mold 100 for the preparation of a hip prosthesis 106. Examples of silicone rubber useful in the present invention include dimethyl silicone rubber, methyl phenyl silicone rubber, methyl vinyl silicone rubber and fluoride silicone rubber. Fumed silica or precipitated silica can be used as a silica-based reinforcing filler for increasing the strength of the mold. Organic peroxide is typically used as a curing agent. The kind of curing agent determines the method and temperature of molding.

As for liquid silicone rubber, a polymer thereof may be compounded with a crosslinking agent, a curing catalyst, a filler, and additives. The type of the liquid silicone rubber determines the available polymers and additives. That is, the kinds of polymers, crosslinking agents, curing catalysts and fillers useful in the present invention may vary depending on whether the liquid silicone rubber is a condensed type, an additive type, a single-component type, or a multi-component type. Available polymers include dihydroxy-polydiethyl-poly-siloxane and divinyl-poly-dimethyl-poly-siloxane. A SiH compound can be used as a crosslinking agent. Examples of the curing catalyst useful in the present invention include organic tin compounds, organic titanium compounds and platinum compounds. As a filler, fumed silica, quartz powder, calcium carbonate, precipitated silica or diatomite may be used. Optionally, additives, such as an adhesion enhancer, a preservative, and a curing controller, may be employed. In this regard, products produced from suitable combinations of polymers, crosslinking agents, curing catalysts, fillers, etc. are commercially available. These commercially available products can be employed in the present invention. Although it is curable rubber, liquid silicone rubber can be continuously supplied to an injection-molding machine, thanks to the liquid property thereof, so that the two-part mold 100 for the preparation of a hip prosthesis 106 can be automatically produced. Particularly, the liquid silicone rubber is outstanding in electrical insulation and weather resistance, in addition to showing flexible properties and characteristic silicon properties over a wide temperature range, from −70 to 200° C. Hence, it is preferable to prepare the two-part mold 100 for the preparation of a hip prosthesis 106 from liquid silicone rubber.

Conventional silicone molding methods, exemplified by silicone compression molding, injection molding, extrusion molding, calendar molding, coating molding and roll molding, can be applied to the preparation of the mold according to the present invention.

The mold for the preparation of recipient blocks in accordance with the present invention can be produced according to various methods, depending on the type of silicone and corresponding molding methods. In an embodiment of the present invention, the mold for the preparation of recipient blocks is prepared by a method comprising (1) charging silicone into a structure having a mold in the form of a a hip prosthesis 106; (2) curing the silicone; and (3) separating the silicone mold for the preparation of hip prosthesis 106 from the structure.

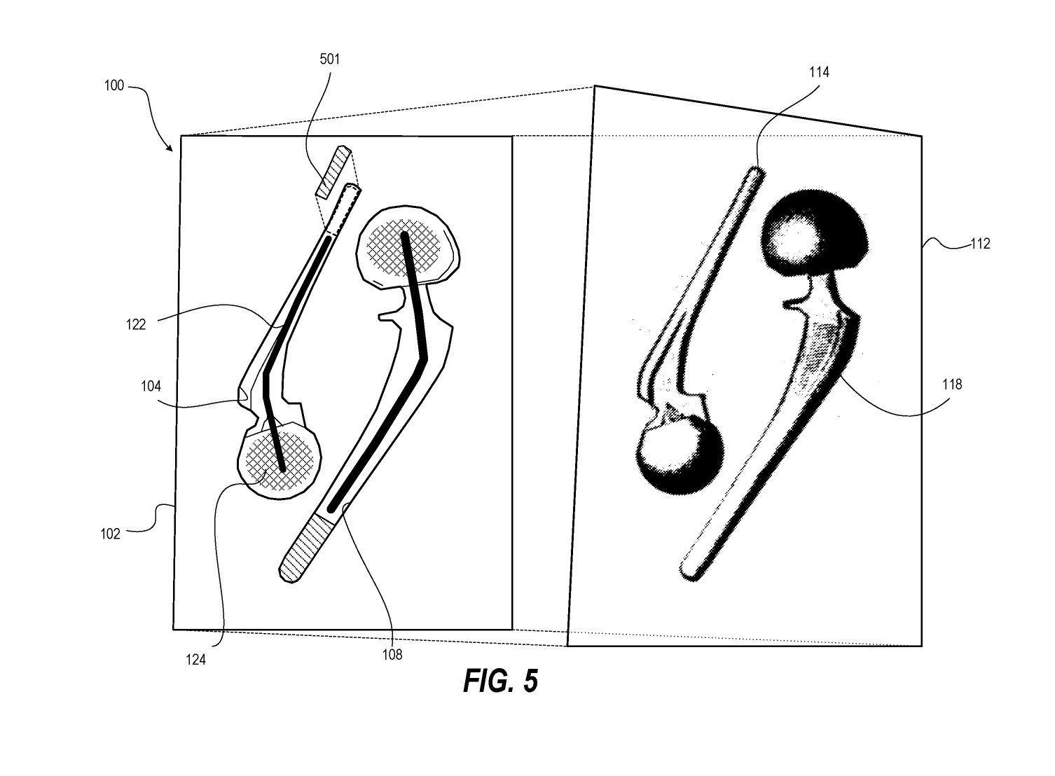

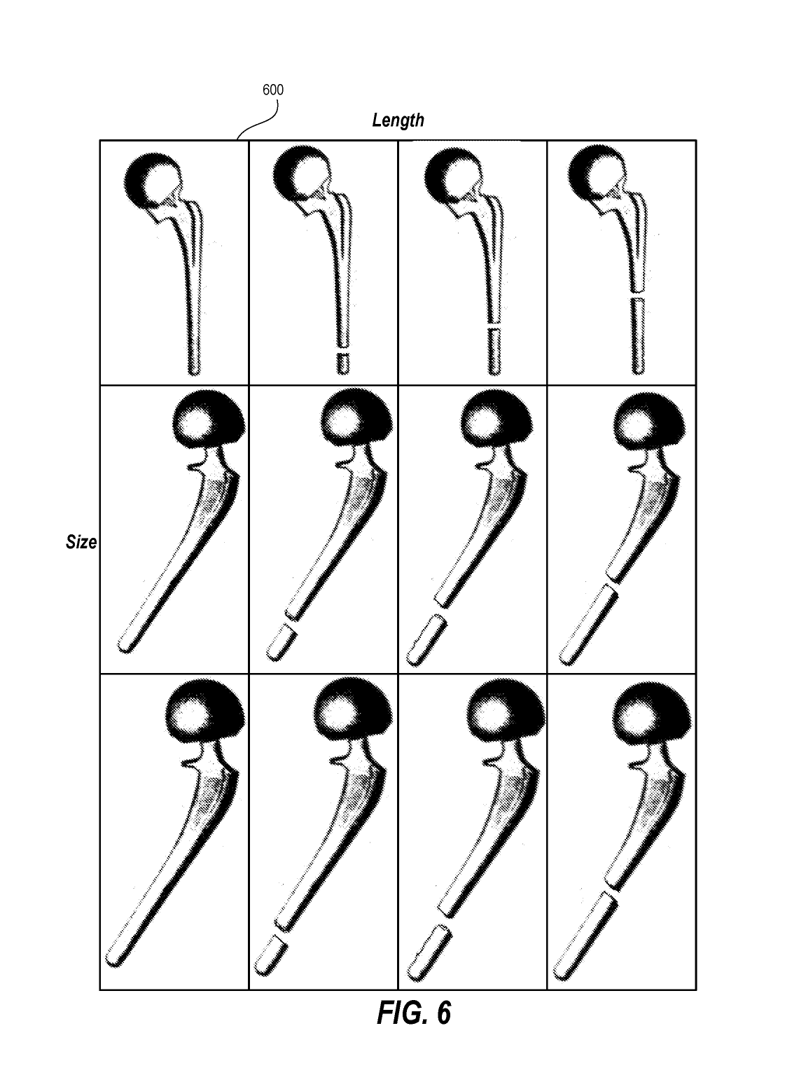

FIG. 5 illustrates inserting a spacer 501 that is sized to fill a tapered end of the front and back impressions 104 of the two-part mold 100. The spacer 501 can be one of a set of varying lengths. For example, FIG. 6 illustrates a data structure 600 that can be referred to select an appropriate mold size and spacer length to achieve a desired prosthesis.

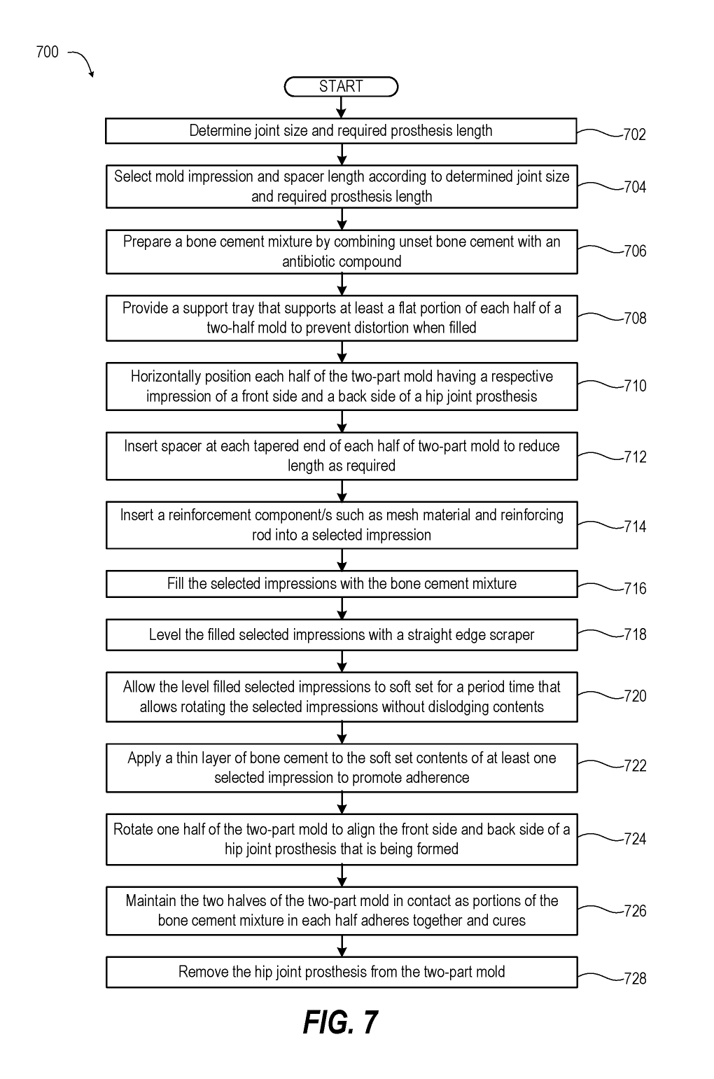

FIG. 7 illustrates a method 700 of making a temporary hip joint prosthesis. In one or more embodiments, the method 700 includes determine a joint size and required prosthesis length (block 702). The method includes selecting a mold impression and spacer length according to the determined joint size and required prosthesis length (block 704). preparing a bone cement mixture by combining unset bone cement with an antibiotic compound (block 706).

In general, a surgeon or assistant will mix the appropriate antibiotic loaded cement or add an antibiotic to the particular cement. It is understood that the preparation of the cement is performed according to the label instructions of the particular cement. For example, about two grams of antibiotic are mixed with each 40-gram packet of bone cement powder, which is then mixed with a corresponding number of 20-milliliter ampoules of a liquid monomer. The bone cement can be a poly-methyl-methacrylate (PMMA) cement such as those produced under the trade names Generation 4™, CMW₁, CMW₂, CMW₃, Zimmer Dough Type, or Zimmer LVC, or a MMA-styrene copolymer cement such as that produced under the trade names Howmedica, Simplex P, or Zimmer Osteobond, or an MMA-methyl acrylate copolymer such as that produced under trade names Cobalt™ G-HV or Cobalt™ HV sold by Biomet. Once the appropriate antibiotic loaded bone cement is mixed, the bone cement is put within a delivery device, such as a cement gun. It is understood that an adaptor may be employed to accommodate different types of delivery devices or cement guns.

The particular antibiotic materials or medicines can be custom selected for a diagnosed type of infection or be a multi-spectrum compound.

In one or more embodiments, the hip prosthesis 106 may include one or more reservoirs to allow for high local concentrations of antibiotics or other specific pharmaceuticals. In one or more embodiments, pharmaceutical-filled hollow fixation pins, in which part of the length has been made capable of drug release, may be used. In one or more embodiments, the pharmaceuticals used are in tablet form, powder form, liquid form, or combinations thereof to allow for graded and timed delivery of the particular medication. In one or more embodiments, the antibiotic delivery involves making beads or using a powdered, stable antibiotic mixed with stabilizing compound such as a common bone cement before being formed into small beads. The antibiotic beads then are packed into the prosthesis 106 for eluting high concentrations of antibiotic over time. In one or more embodiments, the pharmaceuticals have refillable potential through conduit to an external out-of-body or internal within body reservoir.

In one or more embodiments, the two-part mold 100 includes strips or particular forms of the cement that are internally associated with suture mesh or webbing that would allow removal from the molding in a semi-solid state and folding or shaping to surfaces of bone or around soft tissue.

The method 700 includes providing a support tray that supports at least a flat portion of each half of a two-half mold to prevent distortion when filled (block 708). The method 700 includes horizontally positioning each half of the two-part mold having a respective impression of a front side and a backside of a hip joint prosthesis (block 710). The method 700 includes inserting the spacer, if any, at each tapered end of each half of the two-part mold to reduce length as required. The method 700 includes inserting a reinforcement component/s such as mesh material and reinforcing rod into a selected impression (block 714).

Exemplary metals for the reinforcement component/s include stainless steel, titanium, cobalt, and the like and various alloys thereof.

In one or more embodiments, the reinforcing component may be a conformal filling material, such as, for example, a liquid plastic resin (e.g., urethane, ABS, PVC, or epoxy), or it may be a plastic foam, such as urethane, polyurethane, styrene, or polystyrene. Alternatively, the reinforcing component may be a non-resin bulk material, such as plaster, cement, or a silicone-based putty or gel, which is cured by simply by drying. Still another alternative for the reinforcing component is a material with a temperature-dependent viscosity; i.e., a material that is a liquid when heated to an elevated temperature, and that thickens to a highly viscous semi-liquid or paste when cooled to ambient (“room”) temperature. Examples of such materials are waxes (both petroleum-based and “natural,” such as beeswax), and certain plastic resins, such as “HYDROPLASTIC” brand thermoplastic, available from TAK Systems, of Wareham, Mass.

In one or more embodiments, for the reinforcing rod, an appropriately sized section of metal bar is cut. For some applications, this bar or “rebar” can be precurved to better fit in a series of hip mold segments or the like. Then, using the preferred member centralizing (or holding) methods and other mold assembly steps described below, the whole mold assembly can be held in place by, for example, insulated vacuum packing, and a premixed quantity of PMMA or other bone cement poured in.

The present method accommodates several sizes of femoral head circumferences. With segmentation, attending surgeons have the luxury of fitting patients with a variety of lower bone stem sizes (i.e. diameters AND lengths) and shapes. Similarly, for the knee, the method and mold system of this invention allow surgical teams to build customized replacement body parts that have a method of constraint. As such, the risks of separation (i.e. dislocation) between femoral and tibial knee implants are reduced. Since all of the mold segments are modular and interchangeable, the entire skeleton can be “built” with said assembly. Thus, the knee and tibia, down to the ankle can be manufactured in the operating room proper. And because of the uniformity in mold segment manufacturing, and relative costs for same, the method of this invention will further encourage “one time” usage of mold segments, further eliminating the need for cleaning and re-sterilization of mold parts otherwise marked for reuse.

The preferred methods of making mold segments by this invention allow surgical staffs to keep low inventories of segmented mold parts on hand. For a typical temporary hip replacement surgery, for example, operating rooms would need to have access to only twelve varieties of hip head sizes, two different stem lengths and five stem diameters along with 1-2 cm connector body increments (five in total) for a total hip inventory of roughly twenty segmented parts. Many of those same parts have applicability in some knee replacement surgeries. For the latter, an additional inventory of knee-specific, mold segments would require keeping on hand: three standard femoral and tibial sizes; three modular femoral and tibial sizes, along with the two stem lengths and five diameters affiliated with typical hip joint implant surgeries.

The fitting of hip, knee and other replacement joints need not be 100% perfect. Should the exteriors to these mold segmented-derived implants have superficial cracking or pitting, in non-stress bearing areas, defects such as those can be kept uncorrected; or they can be aesthetically patched using the typical cement mold sculpting instruments found in many surgical operating rooms today.

While the modular mold segments of this invention include an open channel or vessel through which bone cement is poured, and through which a reinforcement member is fitted during the mold manufacturing process, these molds do not require any air venting ports like those shown and described by Smith et al. Nor does the present method of mold manufacture hereby require footplates for leveling a mold during cement filling. A vacuum drawn, holding bag is sufficient substitution for leveling these molds while liquidous cement is poured (or ladeled in) and allowed to chemically cure. Alternatively, the cements of these molds can be mixed in small quantities and added to a caulking-style cement gun. Either way, it is preferred that at least some initial quantity of bone cement be added to the combination of connected mold segments before the reinforcement member gets added through the channels and into the mold segments proper. When time is not of the essence, it is even conceivable to add bone cement to the molds of this invention in discretely distinct layers.

For making a temporary hip implant by the present invention, it is first necessary for the surgeon or another member of the surgical team to first “size” the patient using a set of trial fittings (not shown) for approximating which sizes of mold segments to first assemble together. Separately, or even concurrent with initial sizing, one or more packets of bone cement powder are next mixed together with the preferred antibiotic(s) for the patient's particular infection-fighting needs.

One line of cement products is the poly-methyl-methacrylate (or PMMA) commonly sold under such present-day trade names as CMW₁, CMW₂, CMW₃, Zimmer Dough Type, or Zimmer LVC. An alternative cement to use is the MMA-styrene copolymer cement made as sold as Howmedia Simplex P or Zimmer Osteobond. Yet another is the MMA-methyl acrylate copolymer variety sold under the Palacos R label. One representative antibiotic suitable for use the foregoing cement lines is a gentamicin. After the one or more antibiotics are blended in, the ampoule of active liquid monomer gets added to the aforementioned and preferably stirred to accelerate the start of cement curing to a limited degree.

The method 700 includes filling the selected impressions with the bone cement mixture (block 716). The method 700 includes leveling the filled selected impressions with a straight edge scraper (block 718). The method 700 includes allowing the level-filled selected impressions to soft set for a period time that allows rotating the selected impressions without dislodging contents (block 720). If required, the method 700 includes applying a thin layer of bone cement to the soft set contents of at least one selected impression to promote adherence (block 722). The method 700 includes rotating one half of the two-part mold to align the front side and back side of a hip joint prosthesis that is being formed (block 724). The method 700 includes maintaining the two halves of the two-part mold in contact as portions of the bone cement mixture in each half adhere together and cures (block 726). The method 700 includes removing the hip joint prosthesis from the two-part mold (block 728).

For clarity, a hip joint is depicted as being formed from two half-impressions. The present innovation can include molding portions of a joint prosthesis that are assembled after molding. The impressions can be of multiples sizes, selectable to approximate the size of the joint of the recipient. The joint prosthesis can be a hip prosthesis as illustrated. Aspects of the present innovation are also applicable to shoulder, knee and elbow joints as well. In addition, other biocompatible materials can be used. Modularity of the molds can also accommodate recipients with varying sizes of each portion of the prosthesis. For example, a length of a middle or tapered section can be selected for a required length with a head size being independently selected.

In the above-described flow chart of FIG. 5, the method may be embodied in an automated manufacturing system that performs a series of functional processes. In some implementations, certain steps of the methods are combined, performed simultaneously or in a different order, or perhaps omitted, without deviating from the scope of the disclosure. Thus, while the method blocks are described and illustrated in a particular sequence, use of a specific sequence of functional processes represented by the blocks is not meant to imply any limitations on the disclosure. Changes may be made with regards to the sequence of processes without departing from the scope of the present disclosure. Use of a particular sequence is therefore, not to be taken in a limiting sense, and the scope of the present disclosure is defined only by the appended claims.

Also provided for are kits comprising the materials necessary for the mold and methods disclosed herein. The kit can include at least one head component mold 12 and at least one stem component mold. In various embodiments, the kit contents can be provided in differing sizes to allow for implant customization. Any combination of features and parts as detailed herein can be included in the kit, such as, for example, an inclusion of a variety of differing surface area increasing features being included on the different components or a deliver device(s). The components of the kit can be individually seated with the outer container. Minor modifications and inclusions in the kit, which are incidental to surgical methods, such as scalpels, antibiotics, cement, gauze, etc. are also included within the scope of the present teachings.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

What is claimed is:

1. A method of making a temporary hip joint prosthesis, the method comprising:
horizontally positioning each half of a two-part mold having a respective impression of a front side and a backside of a hip joint prosthesis;
level filling the selected impressions with a bone cement mixture;
rotating one half of the two-part mold to align the front side and back side of the hip joint prosthesis;
maintaining the two halves of the two-part mold in contact as portions of the bone cement mixture in each half adheres together and cures; and
removing a hip joint prosthesis from the two-part mold.

2. The method of claim 1, further comprising, prior to level filling the selected impressions, inserting a reinforcement component into a selected impression.

3. The method of claim 2, wherein the reinforcement component comprises a mesh material.

4. The method of claim 2, wherein the reinforcement component comprises a reinforcement rod.

5. The method of claim 1, further comprising preparing the bone cement mixture by combining unset bone cement with an antibiotic compound.

6. The method of claim 1, wherein level filling the selected impressions comprises:
dispensing an amount of the bone cement mixture that is sufficient to fill the selected impression; and
moving a straight edge scraper across the selected impression to remove any excess bone cement mixture from the half of the two-part mold.

7. The method of claim 1, wherein each half of the two-part mold comprises a respective impression of a front side and a back side of a first hip joint prosthesis of a first size and comprises a respective impression of a front side and a back side of a second hip joint prosthesis of a second size, the method further comprising:
level filling at least one of the selected impressions of the first and second hip joint prostheses with a bone cement mixture; and
rotating one half of the two-part mold to align the front side and back side of the respective impressions of both the first and second hip joint prostheses.

8. A method of making a temporary joint prosthesis, the method comprising:
horizontally positioning each half of a two-part mold having a respective impression of a front side and a backside of at least a portion of a joint prosthesis;
level filling the selected impressions with a bone cement mixture;
rotating one half of the two-part mold to align the front side and back side of the hip joint prosthesis;
maintaining the two halves of the two-part mold in contact as portions of the bone cement mixture in each half adheres together and cures; and
removing the at least a portion of a joint prosthesis from the two-part mold.

9. The method of claim 8, wherein each half of the two-part mold further comprise a respective impression of a front side and a back side of another portion of the joint prosthesis, the method further comprising:
level filling at least one of the selected impressions of the portions of the hip joint prostheses with a bone cement mixture;
rotating one half of the two-part mold to align the front side and back side of the respective impressions of both portions of the joint prostheses; and
after removing the portions of the joint prosthesis from the two-part mold, attaching the portions to form a complete joint prosthesis.

10. The method of claim 8, wherein the joint prosthesis is a hip joint prosthesis.

11. The method of claim 8, wherein the joint prosthesis is a shoulder joint prosthesis.

12. The method of claim 8, wherein the joint prosthesis is an elbow joint prosthesis.

13. The method of claim 8, wherein the joint prosthesis is a knee joint prosthesis.

14. The method of claim 1, further comprising:
inserting a spacer at a tapered end of the mold prior to add bone cement; and
detaching the spacer from the completed joint prosthesis.
